(19) United States
(12) Patent Application Publication
(10) Pub. No.: US 2025/0298040 A1
(43) Pub. Date: Sep. 25, 2025

(54) MEASUREMENT OF REACTIVE OXYGEN SPECIES TO ASSESS RED BLOOD CELL QUALITY

(71) Applicant: UNIVERSITY OF MARYLAND, BALTIMORE, Baltimore, MD (US)

(72) Inventors: Joseph P.Y. Kao, Silver Spring, MD (US); Paul W. Buehler, New Market, MD (US); Eric A. Legenzov, Baltimore, MD (US)

(21) Appl. No.: 19/223,163

(22) Filed: May 30, 2025

Related U.S. Application Data
(63) Continuation of application No. PCT/US2023/082049, filed on Dec. 1, 2023.
(60) Provisional application No. 63/385,692, filed on Dec. 1, 2022.

(51) Int. Cl. G01N 33/80

(52) U.S. Cl. CPC G01N 33/80; G01N 2800/22

(57) ABSTRACT

The use of a cell-permeant molecular probe to assay the presence, and monitor the production, of the reactive oxygen species, superoxide, in red blood cells (RBCs) is described. The assay is performed using electron paramagnetic resonance (EPR) spectroscopy and a cell-permeant hydroxylamine spin trap. The cell-permeant molecular probe is not detected or quantifiable by EPR in its reduced form. However, upon entering the RBCs, the probe is oxidized by reacting with superoxide to yield a stable nitroxide radical that is detected and quantified using EPR. Our studies indicate that superoxide production is consistent with RBC metabolism that predicts lipid oxidation, membrane injury and poor post-transfusion performance. Therefore, measurement of reactive oxygen species can be used as an indicator of RBC quality at the time of donation and after storage.

### CROSS REFERENCE TO RELATED APPLICATIONS

This application is a continuation application claiming priority to International Patent Application No. PCT/US2023/082049 filed on Dec. 1, 2023, which claims priority to U.S. Provisional Patent Application No. 63/385,692 filed on Dec. 1, 2022 in the name of Joseph P. Y. Kao et al. and entitled “Measurement of Reactive Oxygen Species to Assess Red Blood Cell Quality,” which is hereby incorporated by reference herein in its entirety.

### FIELD

The present invention relates to assays and methods of monitoring the presence and production of reactive oxygen species in red blood cells (RBCs).

### DESCRIPTION OF THE RELATED ART

Standard refrigerator storage causes metabolic, protein and lipid changes in human RBCs. Depending on processing and donor variability, the resulting changes can negatively impact post-transfusion viability of the RBCs [Yoshida T, et al., 2019]. Donor blood processing is a current topic of research interest directed toward optimizing methods to assess post-transfusion effectiveness [Ochocinska M J, et al., 2021; Vostal J G, et al., 2016], while research on donor variability focuses on defining the contributions of donor-specific characteristics, e.g., the exposome, diet, drugs, age, sex, and genetics [D'Alessandro A, et al., 2021; Nemkov T, et al., 2021]. These factors are now known to be cumulative contributors to changes in the molecular, biochemical and physiological quality of RBCs intended for transfusion. Oxidative stress is a major modifier of normal RBC biology. Within the ex vivo setting of refrigerator storage, cumulative oxidative damage may be magnified by impaired or inadequate protective and repair processes [Reisz J A, et al., 2018]. A current hypothesis suggests that, rather than age, progressive aberrant metabolic, protein and lipid changes decrease RBC quality [D'Alessandro A, et al., 2019]. Therefore, new methods for assessing factors that are more determinative of donor blood quality are a logical step toward optimizing blood storage methodology.

The mature RBC population accounts for approximately 70 percent of the total cellular population of a healthy human [Sender R, et al., 2016]. The oxygen (O₂) carrying protein, hemoglobin (Hb), makes up 95 percent of the RBC content [Beutler E W W, et al, 2006]. One unit of packed RBCs for transfusion contains approximately 60 grams of Hb and 210 mg of iron [Agnihotri N, et al., 2014]. Iron must be coordinated within each globin chain's heme to facilitate efficient O₂ transport through allosteric transitioning between oxyHb (HbFe²⁺·O₂) and deoxyHb (HbFe²⁺). Within RBCs, HbFe²⁺·O₂ is involved in a continuous cycle of spontaneous one-electron oxidations that generates superoxide (O₂^{·-}) and metHb

(HbFe³⁺) (FIG. 1A), wherein O₂^{·-} is the origin of reactive species that are generated within the RBC. Autoxidation of erythrocytic Hb proceeds at different rates for the α- and β-chains, and is influenced by Hb concentration, pH, and the presence of allosteric effectors [Tsuruga M, et al., 1997]. According to current knowledge, autoxidation is the only experimentally defined source of O₂^{·-} in stored RBCs. Under conditions of homeostasis, NADH-dependent cytochrome b5 reductase (CB5R, also termed metHb reductase or diaphorase 1) efficiently reduces HbFe³⁺ to HbFe²⁺ to maintain low levels of oxidized Hb, absent congenital CB5R deficiencies [Gibson Q H, et al., 1948]. Additional prooxidants and reactive oxygen species can be generated within RBCs. In addition to autoxidation, refrigerator-stored RBCs are primed for oxidative stress due to impaired generation of antioxidant enzyme cofactors NADH and NADPH through glycolysis and the pentose phosphate pathway (PPP) respectively (FIG. 1A) [Francis R O, et al., 2020; Rogers S C, et al., 2021]. Moreover, RBC characteristics that directly affect energy metabolism in humans (glucose-6-phosphate dehydrogenase (G6PD) deficiency) [Francis R O, et al., 2020; Roubinian N H, et al., 2022] or genetic polymorphisms that enhance oxidative reactions in human RBCs (SEC14L4, HBA2, and MYO9B) [Roubinian N H, et al., 2022] and in FVB mice (six-transmembrane epithelial antigen of prostate (STEAP-3) expression) [Howie H L, et al., 2019] further increase the potential for prooxidant generation and RBC injury.

In the present disclosure, X-band electron paramagnetic resonance (EPR) spectroscopy and cyclic hydroxylamine-based spin probing of murine and human RBCs is used to identify the generation of O₂^{·-} as a predictive biomarker of RBC oxidative stress and quality.

### SUMMARY

In one aspect, a method of monitoring a sample comprising red blood cells (RBCs) for the presence of superoxide (O₂^{·-}) is disclosed, said method comprising:

combining the sample comprising RBCs with a hydroxylamine molecular probe, wherein if the RBCs of the sample comprise O₂^{·-}, stable nitroxides are formed; and

measuring for the presence of stable nitroxides in the sample using electron paramagnetic resonance (EPR), wherein the hydroxylamine molecular probe is EPR silent and the stable nitroxides have a detectable EPR spectral signal, wherein the detectable EPR spectral signal evidences the presence of O₂^{·-} species in the RBCs of the sample.

In another aspect, a method of measuring a concentration of stable nitroxides in a sample comprising RBCs is disclosed, said method comprising:
combining the sample comprising RBCs with a hydroxylamine molecular probe, wherein if the RBCs of the sample comprise O₂^{·-}, stable nitroxides are formed;
measuring for the presence of the stable nitroxides in the sample using electron paramagnetic resonance (EPR), wherein the hydroxylamine molecular probe is EPR silent and the stable nitroxides have a detectable EPR spectral signal;
calculating the concentration of stable nitroxides in the sample; and
comparing the calculated concentration of stable nitroxides in the sample with the regulatory standard value, wherein if the calculated concentration is greater than the regulatory standard value, the RBCs in the sample are considered to be poor quality.

In another aspect, a method of measuring a rate of oxidation of a hydroxylamine molecular probe in the presence of red blood cells (RBCs) is disclosed, said method comprising:
combining a sample comprising RBCs with a hydroxylamine molecular probe, wherein if the RBCs of the sample comprise superoxide (O₂^{·-}), stable nitroxides are formed;
measuring for the presence of stable nitroxides in the sample using electron paramagnetic resonance (EPR), wherein the hydroxylamine molecular probe is EPR silent and the stable nitroxides have a detectable EPR spectral signal;
calculating the rate of oxidation of a hydroxylamine molecular probe in the sample; and
comparing the calculated rate of oxidation in the sample with the regulatory standard value, wherein if the calculated rate of oxidation is greater than the regulatory standard value, the RBCs in the sample are considered to be poor quality.

In yet another aspect, a method of quantitatively determining a steady-state concentration of superoxide (O₂^{·-}) in a red blood cell (RBC) population is disclosed, said method comprising:
combining a sample comprising the RBC population with a hydroxylamine molecular probe, wherein if the RBCs of the sample comprise O₂^{·-}, stable nitroxides are formed; and
measuring for the presence of stable nitroxides using EPR, wherein the hydroxylamine molecular probe is EPR silent and the stable nitroxides have a detectable EPR spectral signal;
calculating the steady-state concentration of O₂^{·-} in the sample; and
comparing the calculated steady-state concentration of O₂^{·-} in the sample with the regulatory standard value, wherein if the calculated steady-state concentration of O₂^{·-} is greater than the regulatory standard value, the RBCs in the sample are considered to be poor quality.

Other aspects, features and advantages of the invention will be more fully apparent from the ensuing disclosure and appended claims.

### BRIEF DESCRIPTION OF THE DRAWINGS

FIG. 1A. Schematic diagram of measurement using CMH. Superoxide (O₂^{·-}) is generated in the RBC. CMH [Dikalov S I, et al., 2018] is membrane-permeant [Dikalov S I, et al., 2011] and freely enters the RBC, where it rapidly reacts with O₂^{·-} to generate a stable/persistent radical CM·. CM· is also membrane-permeant [Shen J, et al., 2006] and thus can freely leave the RBC.

FIG. 1B. CMH is EPR-silent (it has no EPR signal), but reaction with O₂^{·-} generates CM·, which has a robust 3-line EPR spectrum. The peak height of the EPR spectrum can be used to quantify the amount of CM· generated and thus the concentration of CM· produced in the sample.

FIG. 2A. Demonstration that CMH reacts with O₂^{·-} to produce the radical, CM·, which is quantified by EPR spectroscopy.

FIG. 2B. Demonstration that CMH is selective for O₂^{·-} and does not react with hydrogen peroxide.

FIG. 3A. Demonstrates that CMH measurement distinguishes between murine RBCs that store well and that store poorly.

FIG. 3B. Demonstrates that CMH measurement distinguishes human RBCs stored under normoxic and hypoxic conditions.

FIG. 4. Demonstrates that CMH measurement differentiates between RBCs from healthy donors and donors with sickle-cell disease.

FIG. 5. Complete blood count RBC parameters following exposure to CMH. Data shown for vehicle (ddH₂O) (solid bars), 100 μM (dot filled bars), 300 μM (horizontal lines), 1 mM (vertical lines), 3 mM (lines cross hatched left) and 10 mM (lines cross hatched right), data represents mean values from 3 technical replicates, P>0.05.

FIG. 6A. Scanning electron microscopy of damaged RBC morphologies.

FIG. 6B. Scanning electron microscopy of RBCs exposed to CMH. Data shown for vehicle (ddH₂O), 100 μM, 300 μM, 1 mM, 3 mM and 10 mM. Left columns show images of RBCs obtained at 5000× magnification (40-micron scale bar). Right columns show images of RBCs obtained at 12,000× magnification (10 micro scale bar).

FIG. 7. Shear stress ektacytometry of RBCs exposed to CMH. The change in RBC elongation index is shown on a linear plot (left, top) and a log-linear plot (left-bottom). Plotted data is shown for ddH₂O (-, •), 100 μM (---, ∘), 300 μM (⋅⋅⋅, ▪), 1 mM (-⋅-, □), 3 mM (-⋅⋅-, ▴) and 10 mM (•••, Δ). First, Data is plotted for the EI at a shear stress of 5.33 Pa to represent the RBC shear stress in capillary networks, in vivo (top, right). Second, Data is plotted for the EI at a shear stress of 30 Pa to represent the RBC shear stress at maximum conditions, in vitro (bottom, right). Data is shown as the mean with 3 technical replicates at 5.33 Pa and 30 Pa, P>0.05.

FIG. 8. Oxygen equilibrium of RBCs exposed to CMH. The change in oxygen saturation as a function of oxygen partial pressure is shown on the left. The P50 values that from the OEC curves are plotted foron the right. Plotted data is shown for ddH₂O (-, •), 100 μM (---, ∘), 300 μM (•••, ▪), 1 mM (-⋅-, □), 3 mM (-⋅⋅-, ▴) and 10 mM (•••, Δ).

### DETAILED DESCRIPTION, AND PREFERRED EMBODIMENTS THEREOF

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents, and other references mentioned herein are incorporated by reference in their entirety. The materials, methods, and examples disclosed herein are illustrative only and not intended to be limiting.

“About” and “approximately” are used to provide flexibility to a numerical range endpoint by providing that a given value may be “slightly above” or “slightly below” the endpoint without affecting the desired result, for example, +/−5%.

The phrase “in one embodiment” or “in some embodiments” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Sample,” “test sample,” “specimen,” “sample from a subject,” “biological sample,” and “patient sample” may be used interchangeably herein to refer to a sample of blood from a subject.

“Subject” and “patient” as used herein interchangeably refers to any vertebrate, including, but not limited to, a mammal (e.g., a bear, cow, cattle, pig, camel, llama, horse, goat, rabbit, sheep, hamster, guinea pig, cat, tiger, lion, cheetah, jaguar, bobcat, mountain lion, dog, wolf, coyote, rat, mouse, and a non-human primate (for example, a monkey, such as a cynomolgus or rhesus monkey, baboon, chimpanzee, etc.) and a human). In some embodiments, the subject is a human.

As defined herein, “reactive oxygen species” include oxygen-containing free radicals, i.e., molecules bearing unpaired electrons and thus are paramagnetic (e.g., O₂^{·-}, hydroxyl radical (HO·), ascorbate radical), and oxygen-containing molecules that do not bear unpaired electrons and thus are non-paramagnetic, but may react further to generate other free radical species (e.g., hydrogen peroxide (H₂O₂), singlet oxygen, dehydroascorbate).

As used herein, a “disease or hematologic disorder” includes, but is not limited to, sickle cell disease, thalassemia, and hereditary spherocytosis.

As used herein, the term “poor quality” RBCs is known in the field of transfusion medicine and refers to RBCs that will display undesirable red blood cell circulation, suboptimal increase in total hemoglobin, poor perfusion and compromised oxygenation in patients post-transfusion. In some embodiments, cells under oxidative stress are substantially likely to be of poor therapeutic quality. Here, “poor quality” collectively refers to RBCs that do not meet the desired purpose of transfusion, which is to restore tissue oxygenation, remove carbon dioxide, exchange nitric oxide and restore the function of respiring tissues.

As used herein, “regulatory criteria” is the considered the standard of good quality versus poor quality RBCs in a sample. Current FDA regulatory criteria for good quality RBCs in the United States includes two parameters: 1) not more than 1% hemolysis of RBCs at 42 days of storage and 2) a post transfusion recovery of not less than 75% of transfused RBCs at 42 days of storage. Analogously, regulatory criteria can be obtained for other countries in the world. In practice, samples from a population are obtained and the distribution of a measured parameter (e.g., concentration of stable nitroxides, oxidation rate of hydroxylamine molecular probe or concentration of steady state O₂^{·-} in the RBCs) over the population is determined, wherein RBCs that are in compliance with the regulatory criteria and RBCs that are not in compliance with the regulatory criteria may be included in the population. In some embodiments, samples from a population are obtained from subjects known to not have a disease or hematologic disorder and the distribution of a measured parameter (e.g., concentration of stable nitroxides, oxidation rate of hydroxylamine molecular probe or concentration of steady state O₂^{·-} in the RBCs) over the population is determined, wherein RBCs that are in compliance with the regulatory criteria and RBCs that are not in compliance with the regulatory criteria may be included in the population. In some embodiments, using statistics, a ceiling or highest value of the measured parameter (e.g., concentration of stable nitroxides, oxidation rate of hydroxylamine molecular probe or concentration of steady state O₂^{·-} in the RBCs) that is considered to be in compliance with the regulatory criteria is determined, hereinafter referred to as a “regulatory standard value.” Any measured parameters greater than the regulatory standard value indicate that the RBCs in the sample are of poor quality. In some embodiments, using statistics, a range of values of the measured parameter (e.g., concentration of stable nitroxides, oxidation rate of hydroxylamine molecular probe or concentration of steady state O₂^{·-} in the RBCs) that are in compliance with the regulatory criteria are determined, hereinafter referred to as a “regulatory standard range.” Any measured parameters outside of the regulatory standard range indicate that the RBCs in the sample are of poor quality. It should be appreciated by the person skilled in the art that the regulatory criteria may differ from country to country and may even be amended and as such, the regulatory standard value may differ from country to country and/or may need to be amended, as understood by the person skilled in the art.

The quality of refrigerator stored RBCs is dictated by storage solutions, blood donor genetics, and the donor exposome. Improving the quality of RBCs for transfusion by modifying storage conditions or identifying poor quality RBCs prior to transfusion is an ongoing challenge. It is known that RBCs ex vivo have a refrigerated shelf-life of only 42 days. Intracellular biochemical changes are reported to associate with RBC membrane injury. Oxidative modification of lipids and proteins that alter membrane morphology can also induce macrophage clearance of RBCs following transfusion. The production of reactive oxygen species (ROS) such as O₂^{·-} occurs in RBCs primarily because of high concentrations of oxyhemoglobin, but also due to dysfunction or the loss of specific enzymes that maintain homeostasis. Despite the impact of O₂^{·-}, the accurate and rapid measurement of O₂^{·-} has not been studied.

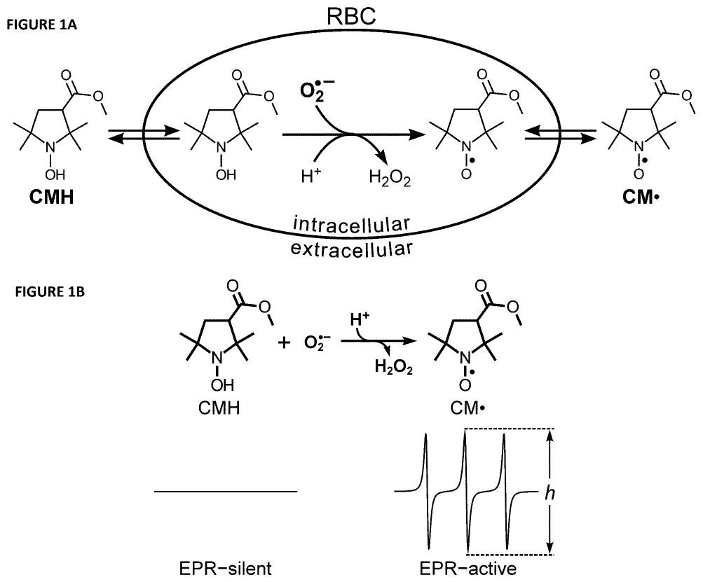

In the present disclosure, it was demonstrated that a hydroxylamine molecular probe and electron paramagnetic resonance (EPR) spectroscopy can be used to quantify O₂^{·-} present in RBCs. The hydroxylamine molecular probe is RBC-permeant and upon entering the RBC, the probe is oxidized by O₂^{·-} present in the RBC to form a stable nitroxide radical (referred to as “stable nitroxides” herein), which is also RBC permeant (see, e.g., FIG. 1). Since hydroxylamine molecular probes are EPR silent and the stable nitroxides have a detectable EPR spectral signal, any O₂^{·-} present in the RBCs manifests as a steady growth of EPR signal. Using this reaction and EPR, refrigerator-stored RBCs can be differentiated between those stored well relative to those stored poorly (e.g., RBCs that do not maintain well in currently available storage solutions, under high oxygen saturations and in response to post-storage irradiation). In addition, the methods described herein can be used to differentiate between human RBCs that have been stored under normoxic conditions from those stored under hypoxic conditions. Still further, the methods described herein can be used to differentiate between RBCs from donors with high-quality and poor-quality RBCs at the time of donation.

In practice, in some embodiments, a concentration of stable nitroxides and O₂^{·-}, using EPR, can be determined as follows. (1) A standard solution of a stable nitroxides, e.g., CM· nitroxide, can be synthesized and confirmed to be analytically pure. It should be appreciated by the skilled artisan that the stable nitroxides synthesized should comprise the stable nitroxides of the hydroxylamine molecular probe chosen for the methods described herein (e.g., CM· nitroxides for CMH). Next, the amplitude of its EPR spectrum is determined, which establishes a calibration between EPR spectral amplitude and the concentration of stable nitroxides, e.g., CM·. (2) The hydroxylamine molecular probe is combined with the sample comprising RBCs and stable nitroxides are generated if O₂^{·-} is present in the RBC, and an EPR spectral amplitude can be obtained. In some embodiments, a typical EPR measurement comprises acquiring spectral amplitudes periodically over time, e.g., about 300 seconds. In some embodiments, “periodically” comprises acquisition of data in intervals ranging from about every 0.1 sec to about every 30 sec, for example every 0.1 sec, every 1 sec, every 2 sec, every 3 sec, every 4 sec, every 5 sec, every 6 sec, every 7 sec, every 8 sec, every 9 sec, every 10 sec, every 15 sec, every 20 sec, every 25 sec, or every 30 sec, as readily determined by the person skilled in the art. (3) A graph of the spectral amplitude of the stable nitroxides, e.g., CM·, versus time can be created, which can be converted to a graph of concentration of stable nitroxides as a function of time using the calibration described in (1). In some embodiments, the graph is a straight line with a measurable upward slope, wherein the slope of the line is the rate of oxidation of hydroxylamine molecular probe into stable nitroxides in the sample. (4) The stoichiometry of O₂^{·-} reaction with the hydroxylamine molecular probe is known to be 1:1, and the rate constant for the reaction can be determined, as understood by the person skilled in the art. The rate of hydroxylamine molecular probe oxidation is Rate=k×[HMP]×[O₂^{·-}]ₛₛ, where k is the rate constant, [HMP] is the concentration of hydroxylamine molecular probe added to the sample of RBCs, and [O₂^{·-}]ₛₛ is the steady-state O₂^{·-} concentration in the sample of RBCs. Since the values of the oxidation rate, k, and [HMP] are all known, [O₂^{·-}]ₛₛ in the sample can be calculated. Since the sample volume occupied by RBCs is the hematocrit (Hct), the average intracellular steady-state O₂^{·-} concentration is therefore [O₂^{·-}]_{SS,RBC}=[O₂^{·-}]_{SS}/Hct.

In a first aspect, a method of distinguishing poor quality RBCs from higher quality RBCs is described. Distinguishing the quality of RBCs, e.g., stored/refrigerated RBCs, is important to ensure that only quality RBCs are administered in future transfusions. Broadly, a hydroxylamine molecular probe is combined with a sample comprising RBCs and if O₂^{·-} are present in the RBCs of the sample, stable nitroxides are generated, which have a robust EPR spectral signal, while the hydroxylamine molecular probe per se is EPR-silent. Advantageously, the presence of an EPR spectral signal, indicating the presence of O₂^{·-} in the RBCs of the sample, can be positively correlated with the quality of the RBCs, wherein poor quality RBCs (e.g., not refrigerated properly, comprise sickle-cells, past the shelf-life) have a higher amount of O₂^{·-} than higher quality RBCs.

In some embodiments, a method of the first aspect relates to monitoring a sample comprising RBCs for the presence of a O₂^{·-}, said method comprising: combining the sample comprising RBCs with a hydroxylamine molecular probe, wherein if the RBCs of the sample comprise O₂^{·-}, stable nitroxides are formed; and measuring for the presence of the stable nitroxides using electron paramagnetic resonance (EPR), wherein the hydroxylamine molecular probe is EPR silent and the stable nitroxides have a detectable EPR spectral signal, and wherein the detectable EPR spectral signal evidences the presence of O₂^{·-} in the RBCs of the sample. In some embodiments, the EPR spectral signal is determined at time t. In some embodiments, the EPR measurement comprises acquiring spectral amplitudes periodically over time. In some embodiments, the method is qualitative. In some embodiments, the method is quantitative wherein a concentration of the stable nitroxides is determined. Too much O₂^{·-} present in the RBCs (e.g., as evidenced by too high of a concentration of stable nitroxides) evidences a blood sample having poor quality that is preferably not used in transfusions or is indicative that the subject suffers from a disease or hematologic disorder. In some embodiments, the method is performed at least 1, 2, 3, 4, or more times, and an average concentration of stable nitroxides is obtained. In some embodiments, the method is performed at least 1, 2, 3, 4, or more times, and an average level of O₂^{·-} is obtained.

In some embodiments, a method of the first aspect relates to measuring a concentration of stable nitroxides in a sample comprising RBCs, said method comprising:
combining the sample comprising RBCs with a hydroxylamine molecular probe, wherein if the RBCs of the sample comprise O₂^{·-}, stable nitroxides are formed;
measuring for the presence of the stable nitroxides in the sample using electron paramagnetic resonance (EPR), wherein the hydroxylamine molecular probe is EPR silent and the stable nitroxides have a detectable EPR spectral signal;
calculating the concentration of stable nitroxides in the sample; and
comparing the calculated concentration of stable nitroxides in the sample with the regulatory standard value, wherein if the calculated concentration is greater than the regulatory standard value, the RBCs in the sample are considered to be poor quality.
   In some embodiments, the EPR spectral signal is determined at time t. In some embodiments, the EPR measurement comprises acquiring spectral amplitudes periodically over time. Too much O₂^{·-} present in the RBCs (e.g., as evidenced by too high of a concentration of stable nitroxides) evidences a blood sample having poor quality that is preferably not used in transfusions or is indicative that the subject suffers from a disease or hematologic disorder. In some embodiments, the method is performed at least 1, 2, 3, 4, or more times, and an average concentration of stable nitroxides is obtained. In some embodiments, the method is performed at least 1, 2, 3, 4, or more times, and an average level of O₂^{·-} is obtained.

In some embodiments, a method of the first aspect relates to measuring a rate of oxidation of a hydroxylamine molecular probe in the presence of RBCs, said method comprising:
combining a sample comprising RBCs with a hydroxylamine molecular probe, wherein if the RBCs of the sample comprise O₂^{·-}, stable nitroxides are formed;
measuring for the presence of stable nitroxides in the sample using EPR, wherein the hydroxylamine molecular probe is EPR silent and the stable nitroxides have a detectable EPR spectral signal;
calculating the rate of oxidation of a hydroxylamine molecular probe in the sample; and
comparing the calculated rate of oxidation in the sample with the regulatory standard value, wherein if the calculated rate of oxidation is greater than the regulatory standard value, the RBCs in the sample are considered to be poor quality.
   In some embodiments, the EPR measurement comprises acquiring spectral amplitudes periodically over time. In some embodiments, a concentration of the stable nitroxides is determined. The higher the rate of oxidation of the hydroxylamine molecular probe in the RBCs, the poorer the quality of the sample comprising RBCs. Poor quality samples are preferably not to be used in transfusions or are indicative that the subject suffers from a disease or hematologic disorder. In some embodiments, the method is performed at least 1, 2, 3, 4, or more times, and an average oxidation rate is obtained.

In a second aspect, a method of quantitatively estimating a steady-state concentration of O₂^{·-} in an RBC population is described, said method comprising:
combining a sample comprising the RBC population with a hydroxylamine molecular probe, wherein if the RBCs of the sample comprise O₂^{·-}, stable nitroxides are formed;
measuring for the presence of stable nitroxides using EPR, wherein the hydroxylamine molecular probe is EPR silent and the stable nitroxides have a detectable EPR spectral signal;
calculating the steady-state concentration of O₂^{·-} in the sample; and
comparing the calculated steady-state concentration of O₂^{·-} in the sample with the regulatory standard value, wherein if the calculated steady-state concentration of O₂^{·-} is greater than the regulatory standard value, the RBCs in the sample are considered to be poor quality.
   In some embodiments, the EPR measurement comprises acquiring spectral amplitudes periodically over time. In some embodiments, an average intracellular steady-state O₂^{·-} concentration is determined. The higher the steady state concentration of O₂^{·-} (or average intracellular steady-state O₂^{·-}) in the RBC population, the poorer the quality of the sample comprising RBCs. Poor quality samples are preferably not to be used in transfusions or are indicative that the subject suffers from a disease or hematologic disorder. In some embodiments, the method is performed at least 1, 2, 3, 4, or more times, and an average steady state concentration of O₂^{·-} is obtained.

A diverse range of cyclic hydroxylamines can be used as the at least one hydroxylamine molecular probe. In some embodiments, the at least one hydroxylamine molecular probe comprises a piperidine derivative, a pyrrolidine derivative, a pyrroline derivative, an oxazolidine derivative, an imidazolidine derivative and/or an imidazoline derivative. In some embodiments, the at least one hydroxylamine molecular probe comprises a compound selected from:

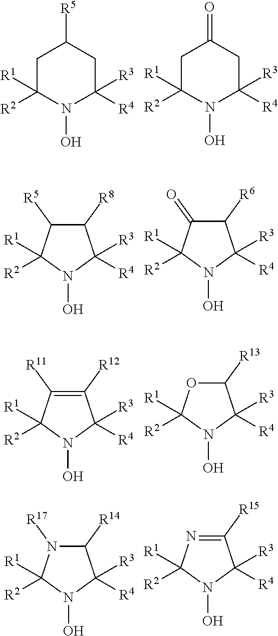

wherein R¹, R², R³, R⁴ can be the same as or different from one another and can be a straight-chained or branched C₁-C₁₀ alkyl group, preferably a straight-chained or branched C₁-C₈alkyl group, more preferably a C₁-C₂ alkyl group;
R⁵ and R⁶ can be the same as or different from one another and can be OR⁷, NR⁸R⁹, or CO₂R¹⁰;
R⁷, R⁸ and R¹⁰ can be the same as or different from one another and can be (i) a straight-chained or branched C₁-C₁₀ alkyl group, preferably a straight-chained or branched C₁-C₈alkyl group, more preferably a C₁-C₂ alkyl group, or (ii) an acyl group comprising 1-10 carbon atoms, preferably 1-5 carbon atoms, more preferably 1-2 carbon atoms;
R¹⁰ can be a straight-chained or branched C₁-C₁₀ alkyl group, preferably a straight-chained or branched C₁-C₅ alkyl group, more preferably a C₁-C₂ alkyl group;
R¹¹, R¹², R¹³, R¹⁴, and R¹⁵ can be the same as or different from one another and can be (i) a straight-chained or branched C₁-C₁₀ alkyl group, preferably a straight-chained or branched C₁-C₈ alkyl group, more preferably a C₁-C₂ alkyl group, or (ii) CO₂R¹⁶; and
R¹⁶ and R¹⁷ can be the same as or different from one another and can be a straight-chained or branched C₁-C₁₀ alkyl group, preferably a straight-chained or branched C₁-C₅ alkyl group, more preferably a C₁-C₂ alkyl group.

In some embodiments, the at least one hydroxylamine molecular probe comprises a piperidine derivative, a pyrrolidine derivative, a pyrroline derivative, an oxazolidine derivative, an imidazolidine derivative and/or an imidazoline derivative. In some embodiments, the at least one hydroxylamine molecular probe comprises a piperidine derivative comprising a structure selected from:

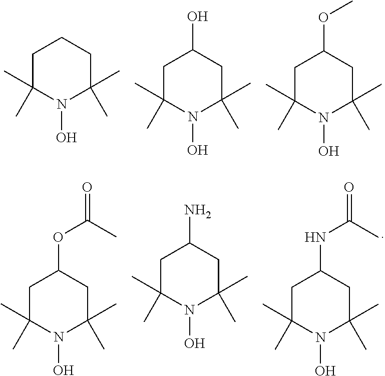

In some embodiments, the at least one hydroxylamine molecular probe comprises a pyrrolidine derivative comprising a structure selected from:

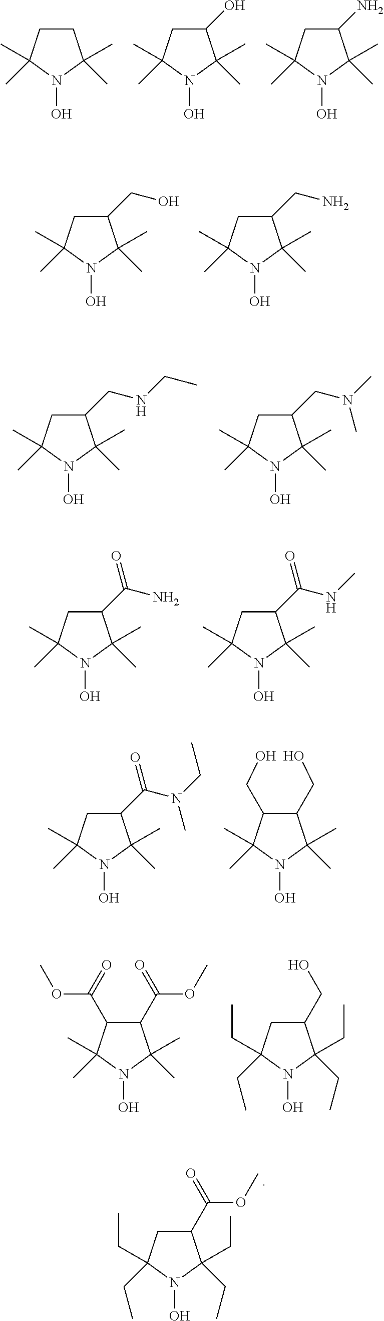

In some embodiments, the at least one hydroxylamine molecular probe comprises a pyrroline derivative comprising a structure selected from:

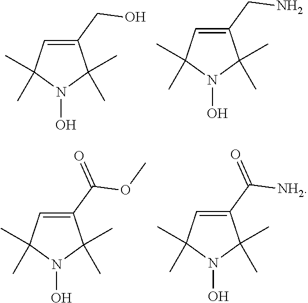

In some embodiments, the at least one hydroxylamine molecular probe comprises an oxazolidine derivative comprising a structure selected from:

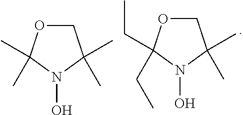

In some embodiments, the at least one hydroxylamine molecular probe comprises an imidazolidine & imidazoline derivative comprising a structure selected from:

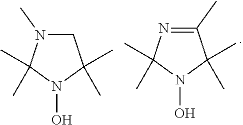

In some embodiments, at least one hydroxylamine molecular probe is used including, but not limited to, 1-hydroxy-3-methoxycarbonyl-2,2,5,5-tetramethylpyrrolidine (CMH), 2-ethyl-1-hydroxy-2,5,5-trimethyl-3-oxazolidine (OXANOH), 4-Hydrazonomethyl-1-hydroxy-2,2,5,5-tetramethyl-3-imidazoline-3-oxide (HHTIO), 1-hydroxy-2,2,5,5-tetramethyl-3-imidazoline 3-oxide (HTIO), 1,3-Dihydroxy-4,4,5,5-tetramethyl-2-(4-carboxyphenyl)tetrahydroimidazole (Carboxy-PTIO-H), 1,4-dihydroxy-2,2,6,6-Tetramethylpiperidine (TEMPOL-H), 1-hydroxy-2,2,6,6-tetramethyl-piperidine (TEMPO-H), 1-hydroxy-2,2,6,6-tetramethyl-4-oxo-piperidine (TEMPONE-H), 1-hydroxy-4-methoxy-2,2,6,6-tetramethylpiperidine (TMH), 1-hydroxy-4-isobutyramido-2,2,6,6-tetramethylpiperidine (TMTH), 1-hydroxy-2,2,6,6-tetramethylpiperidin-4-yl-trimethylammonium (CAT1H), 1-hydroxy-4-phosphono-oxy-2,2,6,6-tetramethylpiperidine (PPH), 1-hydroxy-4-[2-triphenylphosphonio)-acetamido]-2,2,6,6-tetramethylpiperidine (mitoTEMPO-H), 1-hydroxy-2,2,5,5-tetramethylpyrrolidine-3-carboxamide (CMPH), 3-carboxy-1-hydroxy-2,2,5,5-tetramethylpyrrolidine (CPH), 3,4-dicarboxy-1-hydroxy-2,2,5,5-tetramethylpyrrolidine (DCPH), or a salt thereof. In some embodiments, the hydroxylamine molecular probe comprises CMH or a salt thereof.

In some embodiments, the concentration of the at least one hydroxylamine molecular probe in the sample, e.g., upon combination with the RBCs at time 0, is in a range from about 0.1 mM to about 10 mM. In some other embodiments, the concentration of the at least one hydroxylamine molecular probe in the sample is in a range from about 0.3 mM to about 10 mM.

In some embodiments, the samples described herein are withdrawn from a subject. In some embodiments, the sample comprising RBCs is processed to remove at least one of plasma, buffy coats, or both, prior to combination with a solution comprising the hydroxylamine molecular probe. In some embodiments, the sample comprising RBCs is not substantially processed prior to combination with a solution comprising the hydroxylamine molecular probe. In some embodiments, the hematocrit level in the sample comprising RBCs is at least 40% prior to combination with a solution comprising the hydroxylamine molecular probe. In some embodiments, the hematocrit level in the sample comprising RBCs is at least 50% prior to combination with a solution comprising the hydroxylamine molecular probe. In some embodiments, the hematocrit level in the sample comprising RBCs is at least 60% prior to combination with a solution comprising the hydroxylamine molecular probe. In some embodiments, the hematocrit level in the sample comprising red blood cells is at least 70% prior to combination with a solution comprising the hydroxylamine molecular probe. In some embodiments, the sample comprising red blood cells is whole blood. In some embodiments, the sample comprises platelets. In some embodiments, the sample comprising RBCs further comprises at least one preservative solution including, but not limited to, Citrate Phosphate Dextrose (CPD), Citrate Phosphate Dextrose Adenine (CPDA-1), AS-1 (ADSOL), AS-3 (NUTRICEL), AS-5 (OPTISOL), AS-7 (SOLX), Saline Adenine Glucose Mannitol (SAG-M), and Phosphate Adenine Glucose Guanosine Saline Mannitol (PAGGSM). In some embodiments, the sample comprising RBCs further comprises CPDA-1. In some embodiments, when present, the at least one preservative solution is present in an amount from about 10 v/v % to about 20 v/v %.

Electron Paramagnetic Resonance is a technique to derive paramagnetic characteristics of materials by exposing the materials to a combination of magnetic and electromagnetic fields that induces resonance of unpaired electrons within those materials. Discussion of EPR principles and techniques can be found in, for example, J. A. Weil and J. R. Bolton, Electron Paramagnetic Resonance: Elementary Theory and Practical John Wiley & Sons, Applications, 2007; Gilbert et al., Electron Paramagnetic Resonance, Volume 20, The Royal Society of Chemistry, Cambridge UK 2007; A. Schweiger and G. Jeschke, Principles of Pulse Electron Paramagnetic Resonance, Oxford University Press, 2001; and G. R. Eaton, S. S. Eaton, D. P. Barr, and R. T. Weber, Quantitative EPR, Springer Vienna, 2010, all of which are herein incorporated by reference in their entireties.

In some embodiments, the EPR measurement is made at time t=10-450 sec after combination of the sample comprising RBCs with a hydroxylamine molecular probe (wherein combination corresponds to t=0). In some embodiments, the EPR measurement is made at t=10-150 sec after combination. In some embodiments, the EPR measurement is made at t=151-250 sec after combination. In some embodiments, the EPR measurement is made at t=251-350 sec after combination. In some embodiments, the EPR measurement is made at t=351-450 sec after combination.

In some embodiments, the amplitude of the center spectral peak of the EPR spectra is used to calculate the concentration of the stable nitroxide or the oxidation rate. This can be done using methods well known in the art to the skilled artisan. For example, a series of standards can be prepared and a calibration curve obtained and used to calculate the concentration of an unknown. In some embodiments, the EPR measurements are performed at temperatures in a range from about 18° C. to about 25° C. In some embodiments, the EPR measurements are performed at temperatures in a range from about 21° C. to about 24° C. Water does absorb microwaves, so large aqueous samples make tuning of the spectrometer problematic. Therefore, in some embodiments, small volumes of cell suspensions in aqueous media are used. In some embodiments, the volume of sample used is less than about 250 μL. In some embodiments, the volume of sample used is less than about 200 μL. In some embodiments, the volume of sample used is less than about 150 μL. In some embodiments, the volume of sample used is less than about 100 μL. In some embodiments, the volume of sample used is less than about 50 μL.

In some embodiments, instead of EPR, UV-visible spectrophotometry or fluorescence spectroscopy methods of detection are used. The benefits of using fluorescence spectroscopy or spectrophotometry are accessibility and lower cost. Further, probes can be created to allow for equivalent or greater sensitivity than EPR. With regards to a spectrophotometric assay, a molecular probe will react with O₂^{·-} and generate a product whose UV-visible absorption spectrum is different from that of the intact molecular probe. The spectral change can then be used to quantify O₂^{·-}. With regards to a fluorometric assay, a molecular probe reacts with the O₂^{·-} and generates a product whose fluorescence emission is different from that of the intact molecular probe. The change could be in intensity, in the excitation wavelength, in the emission wavelength, or some combination of all three.

The methods described herein are a reliable and simple method to assess whether a sample of blood comprising RBCs is considered to be of poor quality, or not, wherein a poor quality blood sample is preferably not distributed for transfusions. The methods described herein can also be used to determine the severity of a disease or hematologic disorder or determine the need for a transfusion in a subject suffering from the disease or hematologic disorder. The methods described herein are quantitative and noninvasive and can use low-cost instrumentation (e.g., a desktop EPR). The methods are simple and provide rapid data acquisition and analysis, with minimal technical expertise required. Moreover, RBCs of poor quality (e.g., high O₂^{·-} or high probe oxidation rate) can be identified at the time of donation which allows exclusion of that donor unit, which would prevent low-quality blood from entering the blood bank as well as RBCs of poor quality can be identified before transfusion thus preventing transfusion of low-quality RBCs into a patient, thus improving patient outcome.

The present subject matter may be a method, an assay and/or a computer program product. In some embodiments, the computer program product may include a computer readable storage medium (or media) having computer readable program instructions thereon for causing a processor to carry out aspects of the present subject matter.

In practice, when blood donations are obtained from a subject, the blood can be collected in a traditional blood collection bag, but in addition, an additional vial or container of blood is obtained for testing. This ensures that the blood in the blood bag is not compromised in any way. In some embodiments, the additional vial or container comprises at least one probe, and optionally at least one preservative solution, wherein upon collection of blood/RBCs, a rapid analysis of O₂^{·-} content can be performed, either at the time of donation or at some later time prior to transfusion. In addition, this can be used to assess RBC stress in genetic hematological disorders such as sickle cell anemia, thalassemia, hereditary spherocytosis, etc. and acquired hematological disorders such as toxin and toxicant exposure. With proper labeling, after the assay is performed on the additional vial of blood and the blood is considered to have poor quality RBCs, the blood in the blood bag will not be distributed for transfusions. Advantageously, with proper storage, e.g., about 2-6° C., stored blood can be analyzed at any time right before distribution.

The features and advantages of the invention are more fully shown by the illustrative examples discussed below.

### Example

### Methods

General. Chemicals were purchased from commercial vendors and used without further purification. Xanthine oxidase and superoxide dismutase from bovine erythrocytes were purchased from Sigma-Aldrich (St. Louis, MO). High-resolution mass spectra were acquired using an electrospray ionization spectrometer (AccuTOF-CS, JEOL, Peabody, MA). ¹H-NMR spectra were recorded in deuterium oxide on a 400-MHz spectrometer (400 MR, Varian, Palo Alto, CA). EPR spectra were recorded on an X-band spectrometer (EMXnano, Bruker Corp, Billerica, MA). EPR samples were contained in 50-μL borosilicate capillary micropipettes (Drummond Scientific Company, Broomall, PA), whose ends were closed with sealing clay.

Synthesis of 1-hydroxy-3-methoxycarbonyl-2,2,5,5-tetramethylpyrrolidine hydrochloride (CMH HCl salt). 1-Benzyloxy-3-methoxycarbonyl-2,2,5,5-tetramethylpyrrolidine (0.226 g, 0.776 mmol), was dissolved in ethanol (10 mL) in a hydrogenation bottle, to which 5% palladium on activated carbon (143 mg) was added. The mixture was agitated overnight under 27 psi H₂ in a hydrogenation apparatus (Parr Instrument Company, Moline, IL). Thereafter, thin-layer chromatography evidenced complete consumption of starting material. The reaction mixture was filtered through Celite and the filtrate was acidified with concentrated HCl (72 μL). The solvent was removed by rotary evaporation to leave a clear oil which, upon drying under vacuum, crystallized to give a quantitative yield of CMH·HCl. The compound was stored at −20° C. under dry argon. ¹H-NMR (δ): 1.40-1.68 (m, 12H), 2.27-2.50 (m, 2H), 3.41-3.46 (m, 1H), 3.79 (s, 3H). HRMS: [M+H]⁺ C₁₀H₂₀NO₃ requires 202.1443, observed 202.1436.

Measurement of CMH oxidation by EPR spectroscopy. All samples for EPR measurement contained 1 mM CMH. Other reagents, when used, were at the following concentrations: xanthine oxidase (XO), 0.03 U/mL; superoxide dismutase (SOD), 5 μM; hypoxanthine (HX), 0.5 mM; hydrogen peroxide (H₂O₂), 1 mM. Cell-free measurements were made in phosphate-buffered saline (PBS) containing 1 mM diethylenetriaminepentaacetic acid (DTPA). For measurements on RBCs, each sample was made by mixing 2 μL of 50 mM CMH·HCl stock solution with 98 μL of RBC suspension (˜40% hematocrit) in Citrate Phosphate Dextrose Adenine (CPDA-1, Sigma-Aldrich, St. Louis, MO). Time zero (t=0) is the time at which CMH is mixed into the sample. The amplitude (h) of the center peak of the CM· nitroxide EPR spectrum was converted into concentration (C in μM) by using a calibration equation, which for this example was C=(h−0.03308)/0.06461, determined using a series of standard samples.

Murine blood collection and storage. Mouse blood collections were approved by the University of Maryland, Baltimore Institutional Animal Care and Use Committee (IACUC) (protocol #0520008). Processed RBCs from two different mouse strains (C57BL/6J or B6 for short, Jackson Laboratory, Barr Harbor, ME and FVB, Charles Rivers, Worcester, MA) were evaluated in this study. Whole blood (approximately 800-1000 μL per mouse) was collected by cardiac puncture using a 21 G×1 inch needle and 1 mL syringe containing Citrate Phosphate Dextrose solution (CPD) from n=15 B6 and n=15 FVB mice. Blood was pooled within the strains in 15 mL falcon tubes and centrifuged at 750×g for 5 minutes. The plasma and buffy coat were removed. Each aliquot was washed 3 times in 5 mL of phosphate-buffered saline containing 10 mM glucose (PBS-G) with gentle swirling for 3 minutes followed by centrifugation at 750×g for 5 minutes. After the final wash, PBS-G was removed and CPDA-1 (840 μL, 14% of the total volume) was added to RBCs (approximately 6 mL of packed RBCs). Hematocrits were approximately 70% across storage tubes. Finally, preparations were split into 12 equal volumes (500 μL) and stored in 1.7 mL Eppendorf tubes (n=3 per mouse strain per time point) in a refrigerator at 4-6° C. Time zero (t=0) is the time at which CMH is mixed into the sample. Measurements were made on day 1 (immediately after processing) and then on days 2, 4 and 8 after processing such that measurements were made from independent tubes and not a single pooled blood tube.

Data analysis and presentation. Least-squares curve fitting and statistical analysis were performed using OriginPro software (OriginLab Corp., Northampton, MA). Results are presented as mean±SD. Difference between the two strains on the same day was assessed by t-test; within-strain differences over multiple days of storage were assessed through ANOVA.

### Results

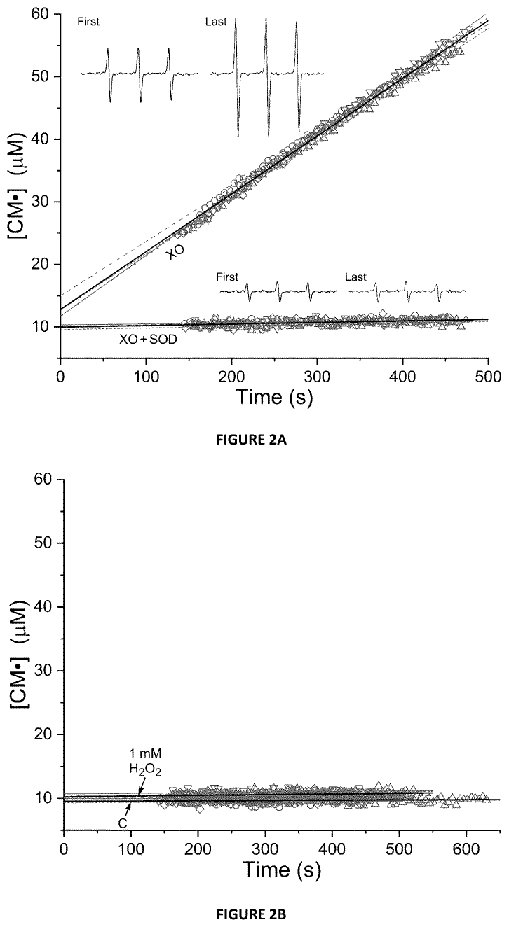

Hydroxylamine molecular probe oxidation by O₂^{·-}. The time course of the reaction of a hydroxylamine molecular probe, e.g., CMH, with O₂^{·-} is shown in FIG. 2A. The enzyme xanthine oxidase (XO) oxidizes its substrate hypoxanthine and generates O₂^{·-} in the process. When CMH is added, reaction with O₂^{·-} converts it to CM·, and the concentration of CM· can be quantified by EPR. The first and last EPR spectrum of a representative data set are plotted as the inset at the top left, showing growth of the EPR spectrum as reaction proceeds. Superoxide dismutase (SOD) destroys O₂^{·-} in a rapid enzymatic process, thus preventing it from reacting with CMH. In a parallel experiment, when CMH is added to a mixture of XO, hypoxanthine, and SOD, negligible production of CM· is observed, indicating that O₂^{·-} is the active species reacting with CMH to generate CM·. For each experiment, 4 replicate measurements are plotted with different symbols; gray lines are least-squares fits to the data; the thick black line is the average of the 4 least-squares lines. For each experiment, the first and last EPR spectrum of a representative data set are plotted as and inset next to the data, showing growth, or lack of growth, of the EPR spectrum with time. Notably, the slope of the line (in units of μM/sec) is the rate at which CMH is oxidized by O₂^{·-}, and the rate is directly proportional to the steady-state O₂^{·-} concentration in the RBCs. Because SOD converts O₂^{·-} to H₂O₂, the XO+SOD result implies that H₂O₂ does not oxidize CMH to CM·. This is confirmed by the experiment shown in FIG. 2B, wherein the incubation of CMH with 1 mM H₂O₂ shows negligible generation of CM·. Since 1 mM H₂O₂ is many orders of magnitude higher than the endogenous H₂O₂ concentration in RBCs, this experiment demonstrates the high selectivity of CMH for O₂^{·-} over H₂O₂. Control experiments (labeled “C”) comprise CMH in buffer without H₂O₂. For each experiment, 4 replicate measurements are plotted with different symbols; gray lines are least-squares fits to the data; the thick black line is the average of the 4 least-squares lines.

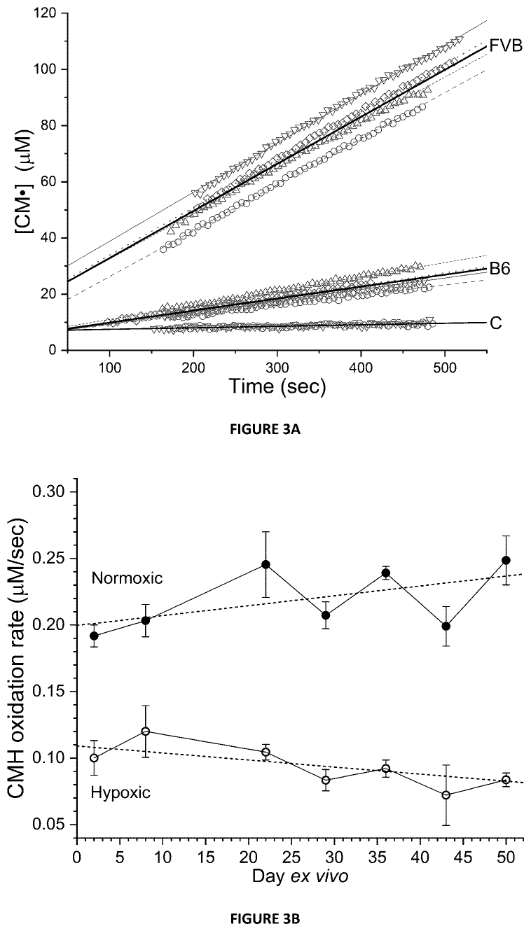

O₂^{·-} generation in red blood cells from FVB and B6 mice during storage. Hydroxylamine molecular probe, e.g., CMH, measurements can be used to distinguish between murine RBCs that store well and that store poorly, as shown in FIG. 3A. RBCs were obtained from two strains of mice: B6, whose RBC store well at refrigerator temperature, and FVB, whose RBCs store poorly at refrigerator temperature. EPR measurement shows that whereas B6 RBCs generate CM· at a low rate (slightly above buffer control CPDA-1, “C”), FVB RBCs generate CM· at a much higher rate. For each RBC experiment, 4 replicate measurements are plotted with different symbols; gray lines are least-squares fits to the data; the thick black line is the average of the 4 least-squares lines; buffer controls comprise duplicate measurements. The measurements were performed on day 2 of RBC storage.

Distinguishing between normoxic and hypoxic storage conditions. Hydroxylamine molecular probe, e.g., CMH, measurements can be used to distinguish human RBCs stored under normoxic and hypoxic conditions, as shown in FIG. 3B. Duplicate samples of human RBCs were stored 1) under normoxic conditions (equilibrated with air), and 2) under hypoxic conditions (equilibrated with low-oxygen atmosphere) and tested periodically with CMH. Hypoxic storage is documented to extend the shelf-life and improve the functional quality of RBCs in storage. CMH measurements show that RBCs stored under normoxic conditions consistently oxidize CMH at a much higher rate than RBCs hypoxically during the entire duration of storage. On any given day of measurement, the normoxic and hypoxic rates are significantly different (p<9.31×10⁻⁴). Each data point is the average of 4-5 replicate measurements; error bars represent s.d.

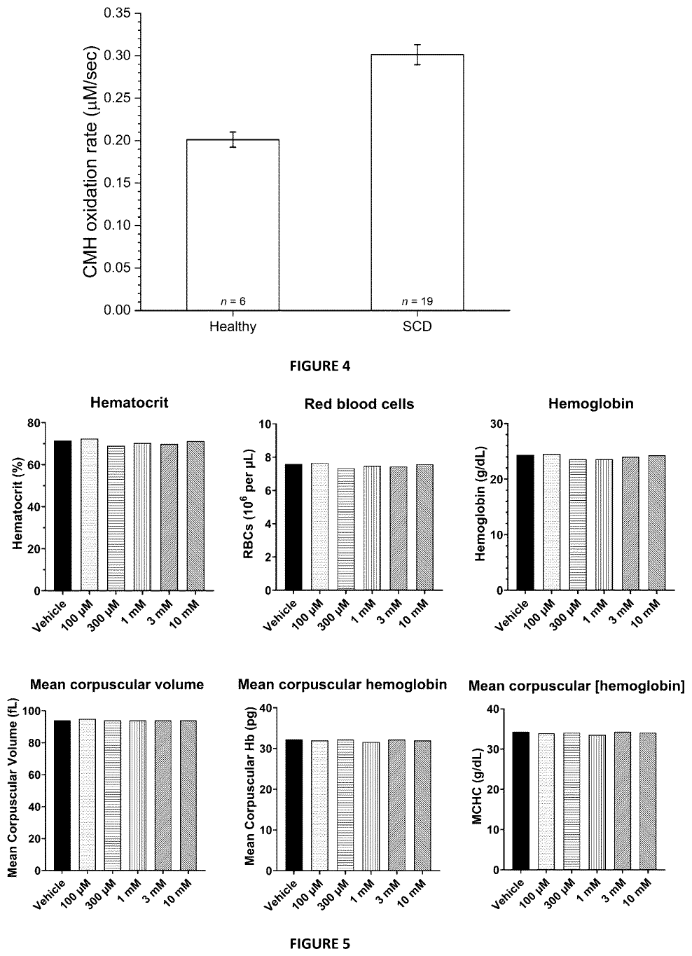

Distinguishing between RBCs from healthy subjects and patients with sickle-cell disease. Hydroxylamine molecular probe, e.g., CMH, measurements can be used to differentiate between RBCs from healthy donors and donors with sickle-cell disease, as shown in FIG. 4. RBCs from screened healthy donors (Healthy, n=6) and donors that have sickle-cell disease (SCD, n=19) were tested with CMH. The CMH measurements show that SCD RBCs clearly oxidize CMH at much higher rates than Healthy RBCs, indicating that the steady-state O₂^{·-} concentration in SCD RBCs is much higher than that in Healthy RBCs. The means of the two populations are significantly different (p=1.26×10⁻⁶). Error bars represent s.e.m.

### Discussion

Refrigerator storage and human donor characteristics alter metabolic processes that induce RBC injury [Reisz J A, et al., 2018; Francis R O, et al., 2020]. From a translational research perspective, murine strains are useful models for blood storage and transfusion experiments. To date, several studies demonstrate that FVB mice produce RBCs with a poor storage phenotype, based on PTR [de Wolski K et al., 2016; Waterman H R, et al., 2015; Kim C Y, et al., 2022]. Current regulatory standards require processed human RBCs meet a 24-hour PTR criterion not less than 75-percent of the infused RBC mass [Vostal J G, et al., FVB mice demonstrate a normal RBC lifespan in vivo [Howie H L, et al., 2019; de Wolski K et al., 2016]; Kim C Y, et al., 2022]; however, storage reduces PTR by as much as 80-percent [de Wolski K et al., 2016]. Further, PTR of stored donor FVB mouse RBCs is associated with metabolic profiles that are consistent with cellular oxidative stress; however, reactive oxygen radicals have not been specifically identified [Id.]. Because of the unique poor storing RBC phenotypic characteristic, the present study was designed to evaluate CMH oxidation to CM· in the RBCs from donor FVB and B6 murine strains on days 1, 2, 4 and 8 of refrigerator storage. This initial proof-of-concept study used X-band EPR measurements to define the potential utility of using CMH oxidation as a probe of O₂^{·-} generation in RBC storage units. It was shown that CMH does not react with hydrogen peroxide, suggesting specificity for reaction with O₂″. Here, stored FVB mouse RBCs showed significantly enhanced rates of CMH to CM· oxidation starting from day 1 of ex vivo storage compared to RBCs from B6 mice. Interestingly, in this study a 3-fold greater mean rate of CMH to CM· oxidation was consistently observed in FVB versus B6 murine RBCs on days 1, 2, 4 and 8 of storage. Further, the mean [CM·] was consistently ˜9 μM higher in RBCs from FVB mice at the time CMH addition on each day of measurement. Both experimental observations identify O₂^{·-} as an important oxygen free radical to generate oxidation products identified in metabolic and proteomic studies of animal and human RBCs under refrigerator storage [Reisz J A, et al., 2018; Wither M, et al., 2016; Stefanoni D, et al., 2020; Thomas T, et al., 2021].

It was therefore demonstrated that a cell-permeant molecular probe (CMH) that is oxidized by O₂^{·-} to generate a stable nitroxide (CM·), in conjunction with a compact X-band EPR spectrometer, can quantify O₂^{·-} generation in RBCs and differentiate murine strains whose RBCs store well or poorly in the refrigerator. This offers a simple method for identifying RBCs of poor quality or at risk of injury prior to transfusion. This method could potentially improve clinical outcomes and reveal determinants of RBC quality that can inform improvements in blood storage technology.

### Example 2

### CMH Concentration Dependent Biocompatibility with Red Blood Cells

### Methods

Donor blood. Blood was obtained from healthy volunteers under informed consent. All human subject research was approved by the University of Maryland, Baltimore Institutional Review Board under Protocol #-HP-00092436.

CMH Incubation. Whole blood was centrifuged at 900 g/2500 rpm for 10 minutes to separate RBC from plasma/buffy coat. Plasma and buffy coat were removed, and RBCs were washed five times with 0.5 ml volumes of isotonic phosphate buffered saline containing 5 mM glucose. After each wash, supernatant and residual buffy coat was removed. After the 5^{th} wash the RBC pellet was mixed with the preservative solution, CPDA-1 in an 85:15 ratio (150 μL CPDA-1 for a total volume of 1 mL). This step was performed to approximate the preparation of packed RBCs for refrigerator storage. CMH was diluted with double deionized H₂O (ddH₂O) to make stock solutions ranging from 400-4 mM. 10 μL of stock CMH solution or ddH₂O was added to 390 μL of RBC/CPDA-1 suspensions, mixed gently but thoroughly and incubated at ambient temperature for 10 minutes. The concentrations tested were as follows: 0.1, 0.3, 1.0, 3.0 and 10 mM. This step was performed to approximate the preparation time needed for assaying superoxide in donor RBCs at the point of collection or in a blood bank setting. After 10 minutes of incubation, complete blood count, shear stress deformability, oxygen equilibrium (oxygen saturation and desaturation) was measured as 3 technical replicates for all samples. Fixation of RBCs was performed for scanning electron microscopy.

Total Blood cell count. Total blood cell count was quantified using ABX Pentra 60 C+ Hematology analyzer (HORIBA Instruments Inc., Kyoto, Japan). Briefly, 60 μL of whole blood was used for the analyses.

Measurements of RBC deformability. RBC deformability was measured by ektacytometry, using the laser-assisted optical rotational cell analyzer (LORCA MaxSis, Mechatronics, Netherlands) at 37° C. The laser integrated device consists of a rotating and a static cylinder that generate shear stresses. Briefly, 2.5-5 mL iso-osmolar Elon Iso solution (Viscosity 28.24 mPa·s, Mechatronics, Netherlands) was mixed with respective volume of the blood sample (based on the RBC count) and placed into the measuring chamber between the two cylinders. RBC deformability was measured by applying 9 different shear stresses (0.30, 0.57, 1.08, 2.04, 3.87, 7.34, 13.92, 26.38, and 30 Pa). A diffraction pattern of RBCs was generated by the laser beam traversing the blood sample. An Elongation Index (EI) was calculated from the diffraction pattern collected by the camera of the LORCA, which reflected RBC deformability, such as: (A−B)/(A+B), with A and B corresponding to the vertical and horizontal axis of a theoretical ellipse fitting the diffraction pattern. Deformability at shear stress equal to 5.33 pascal (Pa) and 30.0 Pa were used for comparisons across groups to approximate shear stress observed the low and at maximum shear, respectively.

Measurement of Oxygen equilibrium (p50). RBCs (based on the RBC count) were added to 3 mL of buffer solution (Hemox, TCS Scientific Corp., New Hope, PA), 6 μL of antifoaming agent (TCS Scientific), and 12 μL of 22% bovine serum albumin (Sigma-Aldrich). The P50 value was determined using an automated blood oxygen analyzer (Hemox, Model B, TCS Scientific) following the manufacturer's protocol. Briefly, samples were oxygenated with air and equilibrated to 37° C., deoxygenated with nitrogen gas, and then an oxygen equilibrium curve was plotted.

Scanning electron microscopy. To prepare cells for scanning electron microscopy (SEM), 25 μL of RBC was suspended in 475 μL of PBS. A solution of 2% glutaraldehyde (Electron Microscopy Sciences Inc., Hatfield, Pennsylvania, USA) in PBS was slowly added to the cell suspension (drop by drop) to reach a final glutaraldehyde concentration of 1%. After incubation for 30 min (RT, dark), the suspension was washed with PBS for 2 min and then centrifuged at 1000×g. 100 μL of the fixed cell suspension was diluted in 400 μL of PBS, and 200 μL of diluted cell suspension was incubated in poly-d-lysine-coated coverslips for 60 min at RT in a six-well plate. Coverslips were rinsed twice with PBS without scratching/damaging the cell layer. After adhesion, coverslips were incubated thrice in 0.1 M cacodylate buffer (Electron Microscopy Sciences) for 10 min at RT. They were then postfixed in 1% osmium tetroxide (Electron Microscopy Sciences) at RT for 60 min. The coverslips were washed twice with Milli-Q water and then dried by incubating them in an ascending series of ethanol (40% to 90%) for 10 min and twice in 100% ethanol for 15 min. Finally, the coverslips were incubated in hexamethyldisilazane (Electron Microscopy Sciences) for 30 min, then dried under a chemical hood. Specimens were coated with Au/Pd (60:40) using a sputter coater (Cressington 108 manual, Cressington Scientific Instruments, Watford, UK) and examined using scanning electron microscopy (SEM) system (FEI Nova NanoSEM 450, Thermo Fisher).

Statistical analysis. Data were processed in Prism (v9.5.1). Data are compared using t-tests with a type 1 error rate (α) of 0.05.

### Results and Discussion

A suite of benchmarking techniques were employed to evaluate the health of RBCs after exposure to CMH at multiple concentrations, up to 10-fold the intended assay concentration. CBC measurements were employed to inform changes in the volume percentage of RBCs, number of RBCs per unit of volume, total concentration of hemoglobin as well as RBC volume and cellular concentrations of hemoglobin. SEM was used to provide visual information about RBC membrane structure. Ektacytometry measurements were made to evaluate the ability of RBCs to elongate under shear stress. These data provide critical information about the cells ability to traverse small blood vessels. Finally, measurements of intra-erythrocytic hemoglobin function were evaluated by decreasing and then increasing oxygen partial pressures in the presence of RBCs. Data from these studies inform the allosteric properties of hemoglobin and the conformational transitioning of globin chains from R (oxy) to T (deoxy) states, which is required to deliver oxygen at peripheral tissue sites and then reoxygenate upon passage through the vasculature of the lungs.

Complete blood counts specific to red blood cell parameters following CMH exposure. A complete blood count was performed after 10 minutes of incubation with CMH at ambient temperature over a range of concentrations (0.1, 0.3, 1.0, 3.0 and 10 mM). Parameters were calculated by standard methodology [Walker H K, et al., 1990]. Samples were prepared as they would be in preparation for blood banking and therefore CBC measurement for RBC parameters represent values for packed RBCs. The effect of up to 10-fold the working concentration of CMH used was tested in the assay. Referring to FIG. 5, it can be seen that no CMH concentration dependent differences were observed in % hematocrit (% of RBCs in circulating plasma), RBC count (μL of RBCs), total hemoglobin (g/dL), Mean corpuscular volume (fL), Mean corpuscular hemoglobin (pg), or mean corpuscular hemoglobin concentration (g/dL). Based on these data, it was concluded that RBCs exposed to CMH at up to 10-fold greater than the intended use in the assay do not impart aberrant physiology based on CBC measurements.

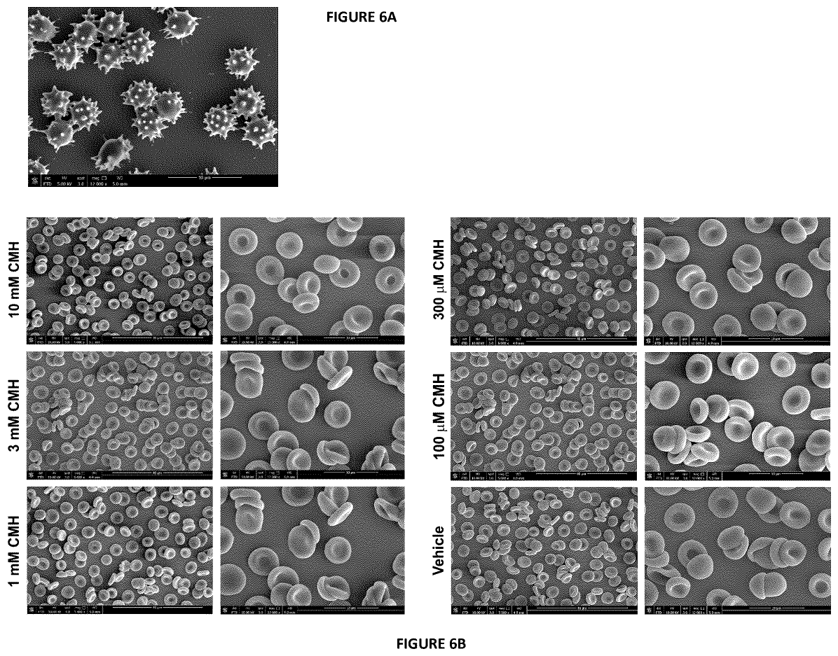

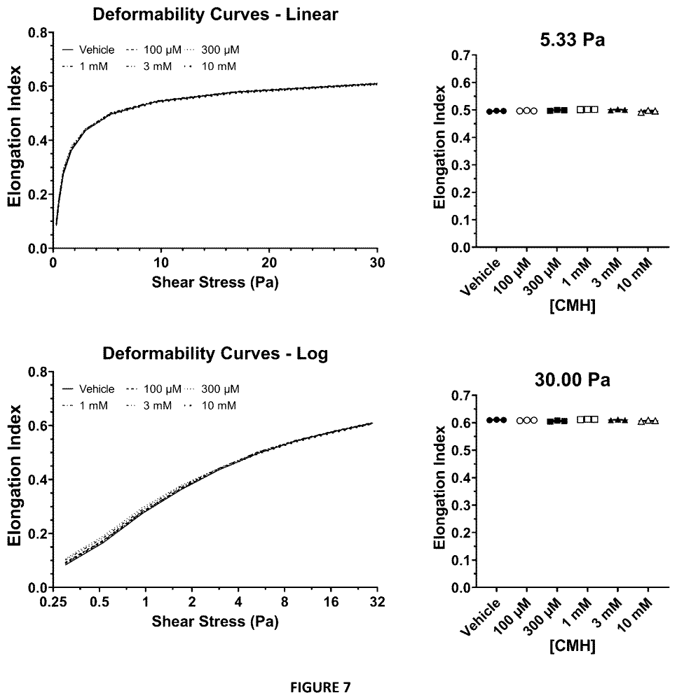

Scanning electron microscopy images of red blood cell following CMH exposure. RBCs were fixed and processed for SEM imaging after 10 minutes of incubation with CMH at ambient temperature over a range of concentrations (0.1, 0.3, 1.0, 3.0 and 10 mM). The morphology of RBCs is an important parameter that helps define their health in disease and after exposure to toxicants and xenobiotics [Farag M R, et al., 2018]. Numerous biochemical changes within RBCs lead to membrane dysfunction and deviations from their normal discoid shape and these membrane specific changes can be visualized effectively using electron microscopy [Geekiyanage N M et al., 2019]. RBC membrane changes were evaluated as a function of CMH concentration. Based on the visual appearance of cells incubated with CMH or vehicle, no remarkable changes to the RBCs, as a function of CMH concentration, were observed as shown in FIG. 6B. This data suggests that RBCs exposed to CMH at up to 10-fold greater than the intended use in the assay do not impart aberrant membrane changes compared to vehicle incubated RBCs.

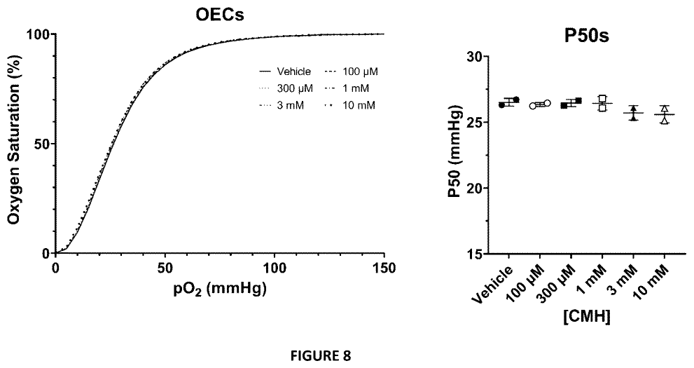

Shear stress ektacytometry red blood cell parameters following CMH exposure. The in vitro measurement of RBC elongation index by shear stress ektacytometry provides critical information about RBC flowability and clearance by macrophages [Mokken F C, et al., 1992]. As shown in FIG. 7, RBCs exposed to CMH at ambient temperature over a range of concentrations (0.1, 0.3, 1.0, 3.0 and 10 mM) do not change the elongation indices of CMH exposed RBCs compared to vehicle exposed RBCs. Elongation index values are plotted at 5.33 Pa to represent the RBC shear stress in capillary networks. Further, data is plotted at a shear stress of 30 Pa to represent the RBC shear stress at maximum conditions, in vitro. This data suggests that RBCs exposed to CMH at up to 10-fold greater than the intended use in the assay do not impart aberrant functional changes in membrane elongation index (flexibility) compared to vehicle incubated RBCs.

Oxygen equilibrium of red blood cell parameters following CMH exposure: Oxygen equilibrium was determined by Hemox analysis [Guarnone R, et al., 1995] over the course of deoxygenation of 100% oxyhemoglobin and then by oxygenation of 100% deoxyhemoglobin, respectively. The first condition was performed starting with 100% oxyhemoglobin followed by switching to 100% nitrogen gas. The concentrations of oxyhemoglobin and deoxyhemoglobin were determined by dual-wavelength spectrophotometry. Following complete oxygen desaturation of hemoglobin, the gas was switched to 100% oxygen, again measuring deoxyhemoglobin and oxyhemoglobin by dual wavelength spectrophotometry. The oxygen equilibrium curves (OECs) and pO₂ (mmHg) at fifty percent oxygen saturation, known as the P50 are shown in FIG. 8. This data suggests that RBCs exposed to CMH at up to 10-fold greater than the intended use in the assay does not impart aberrant hemoglobin allostery expected.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

Agnihotri N, Pal L, Thakur M, Kumar P. The need to label red blood cell units with their haemoglobin content: a single centre study on haemoglobin variations due to donor-related factors. Blood Transfus. 2014; 12 (4): 520-526.
Beutler E W W, Lichtman M, Kipps T J, Kaushansky K, Seligsohn U, Prchal J, Kipps T, Josef P. Williams hematology. McGraw-Hill Professional; 2006. Williams hematology. McGraw-Hill Professional; 2006.
D'Alessandro A, Zimring J C, Busch M. Chronological storage age and metabolic age of stored red blood cells: are they the same? Transfusion. 2019; 59 (5): 1620-1623.
D'Alessandro A, Fu X, Kanias T, et al. Donor sex, age and ethnicity impact stored red blood cell antioxidant metabolism through mechanisms in part explained by glucose 6-phosphate dehydrogenase levels and activity. Haematologica. 2021; 106 (5): 1290-1302.
de Wolski K, Fu X, Dumont L J, et al. Metabolic pathways that correlate with post-transfusion circulation of stored murine red blood cells. Haematologica. 2016; 101 (5): 578-586.
Dikalov S I, Kirilyuk I A, Voinov M, Grigor'ev I A. EPR detection of cellular and mitochondrial superoxide using cyclic hydroxylamines. Free Radic Res. 2011; 45 (4): 417-430.
Dikalov S I, Polienko Y F, Kirilyuk I. Electron Paramagnetic Resonance Measurements of Reactive Oxygen Species by Cyclic Hydroxylamine Spin Probes. Antioxid Redox Signal. 2018; 28 (15): 1433-1443.
Farag M R, Alagaway M. Erythrocytes as a biological model for screening of xenobiotics toxicity, Chem Biol Interact. 2018, 279, 73-83.
Francis R O, D'Alessandro A, Eisenberger A, et al. Donor glucose-6-phosphate dehydrogenase deficiency decreases blood quality for transfusion. J Clin Invest. 2020; 130 (5): 2270-2285.
Geekiyanage N M, Balanant M A, Sauret E, Saha S, Flower R, Lim C T, Gu Y. A coarse-grained red blood cell membrane model to study stomatocyte-discocyte-echinocyte morphologies, PLOS One, 2019, 14 (4), e0215447.
Gibson Q H. The reduction of methaemoglobin in red blood cells and studies on the cause of idiopathic methaemoglobinaemia. Biochem J. 1948; 42 (1): 13-23.
Guarnone R, Centenara E, Barosi G. Performance characteristics of Hemox-Analyzer for assessment of the hemoglobin dissociation curve. Haematologica, 1995, 80 (5), 426-30.
Howie H L, Hay A M, de Wolski K, et al. Differences in Steap3 expression are a mechanism of genetic variation of RBC storage and oxidative damage in mice. Blood Adv. 2019; 3 (15): 2272-2285.
Kim C Y, Johnson H, Peltier S, et al. Deuterated Linoleic Acid Attenuates the RBC Storage Lesion in a Mouse Model of Poor RBC Storage. Front Physiol. 2022; 13:868578.
Mokken F C, Kedaria M, Henny C P, Hardeman M R, Gelb A W. The clinical importance of erythrocyte deformability, a hemorrheological parameter, Ann Hematol. 1992, 64 (3), 113-22.
Nemkov T, Stefanoni D, Bordbar A, et al. Blood donor exposome and impact of common drugs on red blood cell metabolism. JCI Insight. 2021; 6 (3).
Ochocinska M J, Spitalnik S L, Abuhamad A, et al. NIH Workshop 2018: Towards Minimally Invasive or Noninvasive Approaches to Assess Tissue Oxygenation Pre- and Post-transfusion. Transfus Med Rev. 2021; 35 (1): 46-55.
Reisz J A, Nemkov T, Dzieciatkowska M, et al. Methylation of protein aspartates and deamidated asparagines as a function of blood bank storage and oxidative stress in human red blood cells. Transfusion. 2018; 58 (12): 2978-2991.
Rogers S C, Ge X, Brummet M, et al. Quantifying dynamic range in red blood cell energetics: Evidence of progressive energy failure during storage. Transfusion. 2021; 61 (5): 1586-1599.
Roubinian N H, Reese S E, Qiao H, et al. Donor genetic and nongenetic factors affecting red blood cell transfusion effectiveness. JCI Insight. 2022; 7 (1).
Sender R, Fuchs S, Milo R. Revised Estimates for the Number of Human and Bacteria Cells in the Body. PLOS Biol. 2016; 14 (8): e1002533.
Shen J, Liu S, Miyake M, et al. Use of 3-acetoxymethoxycarbonyl-2,2,5,5-tetramethyl-1-pyrrolidinyloxyl as an EPR oximetry probe: potential for in vivo measurement of tissue oxygenation in mouse brain. Magn Reson Med. 2006; 55 (6): 1433-1440.
Stefanoni D, Shin H K H, Baek J H, et al. Red blood cell metabolism in Rhesus macaques and humans: comparative biology of blood storage. Haematologica. 2020; 105 (8): 2174-2186.
Thomas T, Cendali F, Fu X, et al. Fatty acid desaturase activity in mature red blood cells and implications for blood storage quality. Transfusion. 2021; 61 (6): 1867-1883.
Tsuruga M, Shikama K. Biphasic nature in the autoxidation reaction of human oxyhemoglobin. Biochim Biophys Acta. 1997; 1337 (1): 96-104.
Vostal J G, Buehler P W, Gelderman M P, et al. Proceedings of the Food and Drug Administration's public workshop on new red blood cell product regulatory science 2016. Transfusion. 2018; 58 (1): 255-266.
Walker H K, Hall W D, Hurst J W (ed.). Clinical Methods: The History, Physical, and Laboratory Examinations, 3^{rd} ed. Boston, Butterworths, 1990.
Waterman H R, Kapp L M, Howie H L, Hod E A, Spitalnik S L, Zimring J C. Analysis of 24-h recovery of transfused stored RBCs in recipient mice of distinct genetic backgrounds. Vox Sang. 2015; 109 (2): 148-154.
Wither M, Dzieciatkowska M, Nemkov T, Strop P, D'Alessandro A, Hansen K C. Hemoglobin oxidation at functional amino acid residues during routine storage of red blood cells. Transfusion. 2016; 56 (2): 421-426.
Yoshida T, Prudent M, D'Alessandro A. Red blood cell storage lesion: causes and potential clinical consequences. Blood Transfus. 2019; 17 (1): 27-52.

What is claimed is:

1. A method of monitoring a sample comprising red blood cells (RBCs) for the presence of superoxide (O₂^{·-}), said method comprising:
combining the sample comprising RBCs with a hydroxylamine molecular probe, wherein if the RBCs of the sample comprise O₂^{·-}, stable nitroxides are formed; and
measuring for the presence of stable nitroxides in the sample using electron paramagnetic resonance (EPR),
wherein the hydroxylamine molecular probe is EPR silent and the stable nitroxides have a detectable EPR spectral signal, and wherein the detectable EPR spectral signal evidences the presence of O₂^{·-} species in the RBCs of the sample.

2. The method of claim 1, further comprising determining if the RBCs in the sample are considered to be poor quality by:
calculating the concentration of stable nitroxides in the sample, and
comparing the calculated concentration of stable nitroxides in the sample with the regulatory standard value, wherein if the calculated concentration is greater than the regulatory standard value, the RBCs in the sample are considered to be poor quality.

3. The method of claim 1, further comprising determining if the RBCs in the sample are considered to be poor quality by:
calculating the rate of oxidation of a hydroxylamine molecular probe in the sample, and comparing the calculated rate of oxidation in the sample with the regulatory standard value, wherein if the calculated rate of oxidation is greater than the regulatory standard value, the RBCs in the sample are considered to be poor quality.

4. The method of claim 1, further comprising determining if the RBCs in the sample are considered to be poor quality by:
calculating the steady-state concentration of O₂^{·-} in the sample, and
comparing the calculated steady-state concentration of O₂^{·-} in the sample with the regulatory standard value, wherein if the calculated steady-state concentration of O₂^{·-} is greater than the regulatory standard value, the RBCs in the sample are considered to be poor quality.

5. The method of claim 2, wherein the sample comprising poor quality RBCs should not be used in transfusions.

6. The method of claim 2, wherein a subject from which the sample comprising poor quality RBCs was obtained suffers from a disease or hematologic disorder.

7. The method of claim 6, wherein the disease or hematologic disorder is selected from the group consisting of sickle cell disease, thalassemia, hereditary spherocytosis, and combinations thereof.

8. The method of claim 1, wherein the EPR measurement comprises acquiring spectral amplitudes periodically over time.

9. The method of claim 8, wherein the EPR measurement can be made periodically over time between about 10 sec and about 450 sec, or more, after combination of the sample comprising RBCs with a hydroxylamine molecular probe.

10. The method of claim 8, wherein periodically corresponds to intervals ranging from about every 0.1 sec to about every 30 sec.

11. The method of claim 1, wherein the at least one hydroxylamine molecular probe comprises a compound selected from: 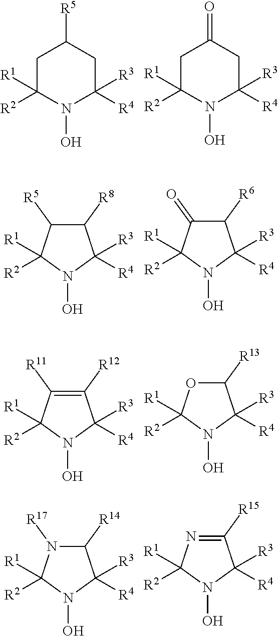
wherein R¹, R², R³, R⁴ can be the same as or different from one another and can be a straight-chained or branched C₁-C₁₀ alkyl group;
R⁵ and R⁶ can be the same as or different from one another and can be OR⁷, NR⁸R⁹, or CO₂R¹⁰;
R⁷, R⁸ and R⁹ can be the same as or different from one another and can be (i) a straight-chained or branched C₁-C₁₀ alkyl group, or (ii) an acyl group comprising 1-10 carbon atoms;
R¹⁰ can be a straight-chained or branched C₁-C₁₀ alkyl group;
R¹¹, R¹², R¹³, R¹⁴, and R¹⁵ can be the same as or different from one another and can be (i) a straight-chained or branched C₁-C₁₀ alkyl group, or (ii) CO₂R¹⁶; and
R¹⁶ and R¹⁷ can be the same as or different from one another and can be a straight-chained or branched C₁-C₁₀ alkyl group.

12. The method of claim 1, wherein the at least one hydroxylamine molecular probe comprises a piperidine derivative, a pyrrolidine derivative, a pyrroline derivative, an oxazolidine derivative, an imidazolidine derivative and/or an imidazoline derivative.

13. The method of claim 1, wherein the at least one hydroxylamine molecular probe is selected from the group consisting of 1-hydroxy-3-methoxycarbonyl-2,2,5,5-tetramethylpyrrolidine (CMH), 2-ethyl-1-hydroxy-2,5,5-trimethyl-3-oxazolidine (OXANOH), 4-Hydrazonomethyl-1-hydroxy-2,2,5,5-tetramethyl-3-imidazoline-3-oxide (HHTIO), 1-hydroxy-2,2,5,5-tetramethyl-3-imidazoline 3-oxide (HTIO), 1,3-Dihydroxy-4,4,5,5-tetramethyl-2-(4-carboxyphenyl)tetrahydroimidazole (Carboxy-PTIO-H), 1,4-dihydroxy-2,2,6,6-Tetramethylpiperidine (TEMPOL-H), 1-hydroxy-2,2,6,6-tetramethyl-piperidine (TEMPO-H), 1-hydroxy-2,2,6,6-tetramethyl-4-oxo-piperidine (TEMPONE-H), 1-hydroxy-4-methoxy-2,2,6,6-tetramethylpiperidine (TMH), 1-hydroxy-4-isobutyramido-2,2,6,6-tetramethylpiperidine (TMTH), 1-hydroxy-2,2,6,6-tetramethylpiperidin-4-yl-trimethylammonium (CAT1H), 1-hydroxy-4-phosphono-oxy-2,2,6,6-tetramethylpiperidine (PPH), 1-hydroxy-4-[2-triphenylphosphonio)-acetamido]-2,2,6,6-tetramethylpiperidine (mitoTEMPO-H), 1-hydroxy-2,2,5,5-tetramethylpyrrolidine-3-carboxamide (CMPH), 3-carboxy-1-hydroxy-2,2,5,5-tetramethylpyrrolidine (CPH), 3,4-dicarboxy-1-hydroxy-2,2,5,5-tetramethylpyrrolidine (DCPH), and any salt thereof.

14. The method of claim 1, wherein the concentration of the at least one hydroxylamine molecular probe in the sample upon combination with the RBCs is in a range from about 0.1 mM to about 10 mM.

15. The method of claim 1, wherein the sample comprising RBCs is selected from (i) whole blood; (ii) samples processed to remove at least one of plasma, buffy coats, or both, prior to combination with a solution comprising the hydroxylamine molecular probe; (iii) samples having hematocrit levels of least 40%; or (iv) samples comprising platelets.

16. The method of claim 1, wherein the samples comprising RBCs further comprise at least one preservative solution selected from the group consisting of Citrate Phosphate Dextrose (CPD), Citrate Phosphate Dextrose Adenine (CPDA-1), AS-1 (ADSOL), AS-3 (NUTRICEL), AS-5 (OPTISOL), AS-7 (SOLX), Saline Adenine Glucose Mannitol (SAG-M), and Phosphate Adenine Glucose Guanosine Saline Mannitol (PAGGSM).

17. The method of claim 1, wherein the volume of sample used is less than 250 μL.

18. The method of claim 1, wherein the EPR measurements are performed at temperatures in a range from about 18° C. to about 25° C.

19. The method of claim 1, wherein the method is used to differentiate between normoxic and hypoxic storage conditions.

20. The method of claim 1, wherein the method is used to differentiate healthy blood donors from blood donors with sickle-cell disease.
